(19) United States
(12) Patent Application Publication
(10) Pub. No.: US 2025/0121199 A1
(43) Pub. Date: Apr. 17, 2025

(54) AUTOMATING DATA DOWNLOAD IN A SYSTEM REQUIRING RESPONSIVENESS TO A SECONDARY CONTROL UNIT

(71) Applicant: Boston Scientific Neuromodulation Corporation, Valencia, CA (US)

(72) Inventors: Lisa Denise Moore, Glendale, CA (US); Chirag Shah, Valencia, CA (US); Raul Enrique Serrano Carmona, Los Angeles, CA (US)

(73) Assignee: Boston Scientific Neuromodulation Corporation, Valencia, CA (US)

(21) Appl. No.: 18/912,023

(22) Filed: Oct. 10, 2024

Related U.S. Application Data
(60) Provisional application No. 63/543,826, filed on Oct. 12, 2023.

(51) Int. Cl. A61N 1/372; A61N 1/36; G16H 40/67

(52) U.S. Cl. CPC A61N 1/37252; G16H 40/67; A61N 1/3605

(57) ABSTRACT

A system for regulating data downloads from an implantable pulse generator (IPG) includes an IPG configured to download data, receive signals, and provide electrical stimulation, and an external device configured to initiate a data download from the IPG and to direct the IPG to download the data for a first time period. The IPG and/or external device is configured to stop the data download after the first time period has expired and to wait a first time interval during which no data is downloaded and the IPG is available to receive signals from any external control devices. The IPG and/or external device is configured to initiate an additional data download from the IPG to the external device for a second time period after the first time interval has expired, and to repeat the steps until all data has been downloaded from the IPG to the external device.

### CROSS-REFERENCE TO RELATED APPLICATIONS

This application claims the benefit of U.S. Provisional Patent Application Ser. No. 63/543,826, filed Oct. 12, 2023, which is incorporated herein by reference.

### BACKGROUND

Implantable and/or wearable stimulations systems for the treatment of various diseases and disorders of the neurological system have proven effective in a wide variety of ways. For example, spinal cord stimulation (SCS) systems are accepted treatments for chronic pain syndromes. Deep brain stimulation (DBS) systems have been used for chronic pain as well, and are gaining acceptance for treatment of movement and tremor disorders. Peripheral nerve stimulation (PNS) systems have also been shown effective for certain indications, and functional electrical stimulation (FES) has been investigated for restoration of functionality to paralyzed extremities.

Some patients may receive a home monitoring system for use with an implantable medical device. The home monitoring system can take the form of a bedside monitor, which will wirelessly communicate with the implantable pulse generator (IPG) of an implantable system. Data collected by the IPG during the day may be download during the evening or overnight while the patient is at rest or asleep by the bedside monitor. Bedside monitors have been widely used with cardiac pacemakers and/or defibrillators. However, with neuromodulation systems (including SCS, DBS, PNS, FES), the patient may receive both a bedside monitor and a patient remote control (RC). The RC may be used to turn therapy on or off, to adjust therapy, and/or to obtain feedback from the patient. There is a potential for conflict between the RC and a bedside monitor if, for example, the bedside monitor is engaged in communication with an IPG at the same time that a patient wishes to use the RC to adjust therapy or turn therapy on or off. For example, absent mitigations, two devices are already engaged in a communication session, a third device may not be allowed to interrupt the ongoing session. New and alternative methods and systems for addressing or mitigating such potential communication conflicts are needed.

### SUMMARY

This disclosure provides design, material, manufacturing method, and use alternatives for medical devices. An example system for regulating data downloads from an implantable pulse generator (IPG) includes an IPG configured to download data, receive operation signals and provide electrical stimulation to a patient, and at least a first external device configured to initiate a first data download from the IPG to the first external device, the first external device configured to direct the IPG to download the data for a first time period, wherein the first time period is preset, wherein the IPG and/or the first external device is configured to stop the data download after the first time period has expired, wherein the IPG and/or the first external device is configured to wait a first time interval during which no data is downloaded and the IPG is available to receive signals from any external control devices, wherein the IPG and/or the first external device is configured to initiate an additional data download from the IPG to the first external device for a second time period after the first time interval has expired, wherein the IPG and/or the first external device is configured to repeat the initiating, stopping, waiting, and initiating additional data download steps until all data has been downloaded from the IPG to the first external device.

Alternatively or additionally to the embodiment above, the first external device is a remote control, bedside monitor, or other device with a monitoring application. Alternatively or additionally to any of the embodiments above, the first external device is configured to communicate with the IPG via a Bluetooth connection. Alternatively or additionally to any of the embodiments above, the IPG and/or the first external device is configured to measure activity level of the patient. Alternatively or additionally to any of the embodiments above, the IPG and/or the first external device includes an accelerometer.

An example method for regulating data downloads from an implantable pulse generator (IPG) includes the steps of initiating a first data download from the IPG to a first external device and downloading data for a first time period, wherein the first time period is preset, stopping the data download after the first time period has expired, waiting a first time interval during which no data is downloaded and the IPG is available to receive signals from any external control devices, after the first time interval has expired, then initiating an additional data download from the IPG to the first external device for a second time period, and repeating the initiating, stopping, waiting, and initiating additional data download steps until all data has been downloaded from the IPG to the first external device. Alternatively or additionally to the embodiment above, the first external device is a remote control for the IPG, a bedside monitor, or other device with a monitoring application. Alternatively or additionally to any of the embodiments above, the first external device is a cell phone. Alternatively or additionally to any of the embodiments above, initiating the first and additional data downloads from the IPG to the first external device occurs over a Bluetooth connection. Alternatively or additionally to any of the embodiments above, the first and second time periods are each between 30 seconds and two minutes. Alternatively or additionally to any of the embodiments above, the first time interval is between 30 seconds and two minutes.

Another example method for regulating data downloads from an implantable pulse generator (IPG) includes the steps of measuring activity level of patient, preventing data download from the IPG to at least a first external device when the activity level has been above a threshold for a first period of time, waiting a second period of time during which no data is downloaded and the IPG is available to receive signals from any external control devices, measuring activity level of the patient, and initiating data download from the IPG to the first external device when the activity level drops below the threshold.

Alternatively or additionally to the embodiment above, the first period of time is at least 30 minutes. Alternatively or additionally to any of the embodiments above, the second period of time is at least 30 minutes. Alternatively or additionally to any of the embodiments above, measuring activity level is achieved by an accelerometer. Alternatively or additionally to any of the embodiments above, measuring activity level is performed by the IPG. Alternatively or additionally to any of the embodiments above, measuring activity level is performed by a wearable device that is in communication, directly or indirectly, with the IPG.

Alternatively or additionally to any of the embodiments above, the method also includes determining if the patient is asleep, and automatically downloading data from the IPG to the first external device when the patient is asleep. Alternatively or additionally to any of the embodiments above, determining if the patient is asleep is achieved by comparing a current time of day with a sleep schedule set by the patient. Alternatively or additionally to any of the embodiments above, determining the patient is asleep is achieved by a lack of activity measured by an accelerometer. Alternatively or additionally to any of the embodiments above, determining the patient is asleep is achieved by measuring the patient's activity on or interaction with all known devices used by the patient.

Alternatively or additionally to any of the embodiments above, initiating the first data download is prevented or stopped for at least a third period of time when a signal between the IPG and the first external device has a strength below a predetermined level. Alternatively or additionally to any of the embodiments above, after the third period of time, if the signal is still below the predetermined level, then stop any attempts to download data for 12 hours. Alternatively or additionally to any of the embodiments above, initiating the first data download is prevented or stopped for at least a third period of time when a battery level of the IPG or the first external device is below a predetermined level.

Alternatively or additionally to any of the embodiments above, if the first data download is prevented after a predetermined number of attempts within a first predetermined period of time, the method further comprises notifying the patient, physician, or caregiver. Alternatively or additionally to any of the embodiments above, when a predetermined number of data downloads have been attempted during a second predetermined period of time, then limiting a number and/or duration of future data downloads in a third predetermined period of time.

The above summary of some embodiments, aspects, and/or examples is not intended to describe each embodiment or every implementation of the present disclosure. The figures and the detailed description which follows more particularly exemplify these embodiments.

### BRIEF DESCRIPTION OF THE DRAWINGS

The disclosure may be more completely understood in consideration of the following detailed description of various embodiments in connection with the accompanying drawings, in which:

FIG. 1 shows an implantable system as a deep brain stimulation (DBS) system;

FIG. 2 shows an implantable system as a spinal cord stimulator (SCS) system; and

FIGS. 3-4 show illustrative methods in block form.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic or optical disks, magnetic cassettes, memory cards or sticks, random access memories (RAMs), read only memories (ROMs), and the like.

The following description should be read with reference to the drawings, which are not necessarily to scale, wherein similar elements in different drawings are numbered the same. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

Current operation of some implantable pulse generators (IPGs) has all external devices that communicate with the IPG having equal priority. In some systems, an IPG may lack a clock that allows time stamping of data, and so a daily product record may be desired to help establish a time course of IPG usage and performance. Downloads of data may be relatively fast under normal conditions (under 5 minutes for example), but could take longer if for example there is a time gap between downloads. Downloads, as used herein, indicates communication of data from the IPG to the remote monitor.

IPGs may be able to download data through a wireless connection, such as a Bluetooth connection, by utilizing available software tools which can be located on a multitude of devices and can be used to manually download data from IPGs. These same software tools may be able to automate data download from IPGs, which may improve the patient experience. However, an automated data download requires one external device to automatically initiate a Bluetooth connection to an IPG, and during this data download connection, a secondary external device, such as the remote control, cannot connect to the IPG. This may create a situation where the remote control is potentially unable to pair with the IPG and therefore unable to change the state of stimulation on the patient in a potentially urgent scenario.

The present inventors have recognized, among other things, that a problem to be solved is preventing interference between data downloads from an IPG and other communication attempts. For example, data downloads to a first external device may interfere with attempted communication from other external devices. The problem to be solved may involve preventing interference between an automatic data download to a first external device and an attempt to connect to the IPG by a second external device. For example, when a separate device from a remote control is connected to the IPG for downloading data, the remote control may be prevented from sending a signal for therapy to the IPG. One solution is to reduce the potential of interference with limited time intervals for the data downloads. Another solution is to initiate downloads while the patient is asleep or at a scheduled time indicated by the patient. Further solutions may involve preventing downloads when the wireless signal is weak (which may extend the communication session due to data transmission retries) and preventing downloads when the battery is low on either the IPG or the external device receiving the download.

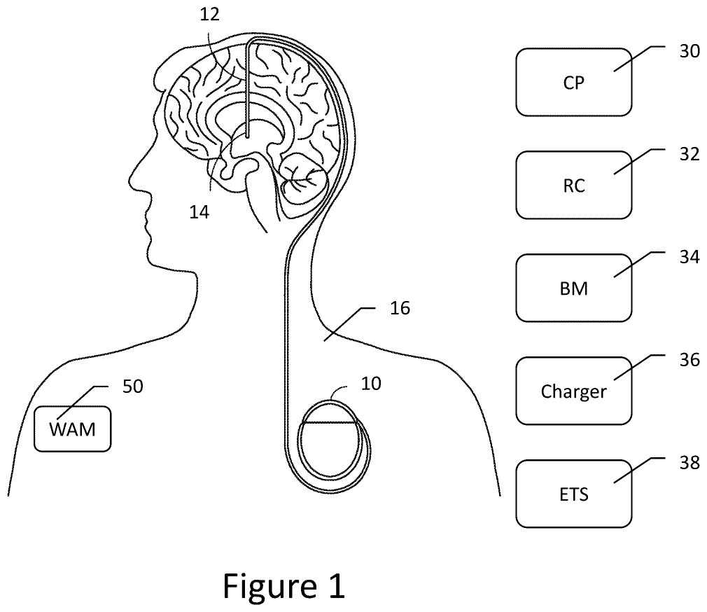

FIG. 1 shows an illustrative DBS system implanted in a patient. The system comprises an implantable pulse generator (IPG) 10, shown implanted in the pectoral region of a patient 16. The IPG 10 is coupled to a lead 12 which extends subcutaneously to the head of the patient 16, through a burr hole formed in the patient's skull, and then into the brain. In the example shown, the lead 12 includes a plurality of electrodes positioned near the distal end 14 of the lead. The lead 12 may be placed at any suitable location of the brain where a target for therapy is identified. For example, a lead 12 may be positioned so that the distal end 14 is near the mid-brain and/or various structures therein that are known in the art for use in providing stimulation to treat various diseases.

DBS may be targeted, for example, and without limitation, at neuronal tissue in the thalamus, the globus pallidus, the subthalamic nucleus, the pedunculopontine nucleus, substantia nigra pars reticulate, the cortex, the globus pallidus externus, the medial forebrain bundle, the periaquaductal gray, the periventricular gray, the habenula, the subgenual cingulate, the ventral intermediate nucleus, the anterior nucleus, other nuclei of the thalamus, the zona incerta, the ventral capsule, the ventral striatum, the nucleus accumbens, and/or white matter tracts connecting these and other structures. Data related to DBS may include the identification of neural tissue regions determined analytically to relate to side effects or benefits observed in practice. “Targets” as used herein are brain structures associated with therapeutic benefits, in contrast to avoidance regions or “Avoid” regions which are brain structures associated with side effects.

Conditions to be treated may include dementia, Alzheimer's disease, Parkinson's disease, dyskinesias, tremors, depression, anxiety or other mood disorders, sleep related conditions, etc. Therapeutic benefits may include, for example, and without limitation, improved cognition, alertness, and/or memory, enhanced mood or sleep, elimination, avoidance or reduction of pain or tremor, reduction in motor impairments, and/or preservation of existing function and/or cellular structures, such as preventing loss of tissue and/or cell death. Therapeutic benefits may be monitored using, for example, patient surveys, performance tests, and/or physical monitoring such as monitoring gait, tremor, etc. Side effects can include a wide range of issues such as, for example, and without limitation, reduced cognition, neuroinflammation, alertness, and/or memory, degraded sleep, depression, anxiety, unexplained weight gain/loss, tinnitus, pain, tremor, etc. These are just examples, and the discussion of ailments, benefits and side effects is merely illustrative and not exhaustive.

The illustrative system described below includes various external devices. A clinician programmer (CP) 30 may be used to determine/select therapy programs. The CP 30 can be used by a physician to manipulate the outputs of the IPG 10 and/or an external test stimulator (ETS) 38. For example, the CP 30 can be used by the physician to define a therapy regimen or program for application to the patient. Multiple programs may be facilitated and stored by the IPG 10 or ETS 38; in some examples, a patient remote control (RC) 32 may store the programs to be used. Communication amongst the IPG 10, RC 32, CP 30, ETS 38 and charger 36 for the IPG may use any suitable protocol such as wireless RF telemetry, inductive communication, Bluetooth, etc.

The CP 30 may be used to determine stimulation parameters. Stimulation parameters may include amplitude of stimulation pulses, frequency or repetition rate of stimulation pulses, pulse width of stimulation pulses, and more complex parameters such as burst definition, as are known in the art. Biphasic square waves are commonly used, though nothing in the present invention is limited to biphasic square waves, and ramped, triangular, sinusoidal, monophasic and other stimulation types may be used as desired. The CP 30 can be used by a physician, or at the direction of a physician, to obtain data from and provide instructions the IPG 10 via suitable communications protocols such as Bluetooth or MedRadio or other wireless communications standards, and/or via other modalities such as inductive telemetry.

The RC 32 can be used by the patient to perform various actions relative to the IPG 10. These may be physician defined options, and may include, for example, turning therapy on and/or off, entering requested information (such as answering questions about activities, therapy benefits and side effects), and making (limited) adjustments to therapy such as selecting from available therapy programs and adjusting, for example, amplitude settings. The RC 32 can communicate via similar telemetry as the CP 30 to control and/or obtain data from the IPG 10. The patient RC 32 may also be programmable on its own, or may communicate or be linked with the CP 30. The RC 32 may be a dedicated device, including a locked off-the-shelf device with specialized software/firmware to prevent other uses, or may be a multi-purpose device such as the patient's cell phone.

A charger 36 may be provided to the patient to allow the patient to recharge the IPG 10, if the IPG 10 is rechargeable. Some IPG 10 are not rechargeable, and so the charger 36 may be omitted. The charger 36 can operate, for example, by generating a varying magnetic field to activate an inductor associated with the IPG 10 to provide power to recharge the IPG battery, using known methods and circuitry.

Some systems may include the ETS 38. The ETS 38 can be used to test therapy programs after the lead 12 has been implanted in the patient to determine whether therapy will or can work for the patient 16. For example, an initial implantation of the lead 12 can take place using, for example, a stereotactic guidance system, with the IPG 10 temporarily left out. After a period of healing, the patient may return to the clinic for therapy configuration and testing. The lead 12 may have a proximal end thereof connected to an intermediate connector (sometimes called an operating room cable) that couples to the ETS 38, and the ETS 38 can be programmed using the CP 30 with various therapy programs and stimulation parameters. Once therapy suitability for the patient is established to the satisfaction of the patient 16 and/or physician, the permanent IPG 10 is implanted and the lead 12 is connected thereto, with the ETS 38 then removed from use.

A remote monitoring system may be included as well. In FIG. 1, the remote monitor takes the form of a bedside monitor (BM) 34. The BM may be a custom or special purpose device or it may be integrated into a standard device, such as a mobile phone, tablet, or the RC 32. In some embodiments, the BM may be an app on a mobile phone, tablet or RC, and the app may be a standalone BM app or the BM functions may be integrated into another app such as an RC app. The BM 34 may download data from the IPG related to the use (such as history of use by the patient), performance, and/or status (such as battery charge state, lead impedance and/or function, etc.) of the IPG. The BM 34 may communicate with other components, such as the RC 32 and/or ETS, as desired. The BM 34 may be in communication with the manufacturer of the IPG, and or with a physician's office, or a central server accessible by one or more of the manufacturers and/or physician. The central server may be in communication with any of the devices disclosed herein, including the BM, RC, CP, wearable activity monitor (WAM), etc. In some examples, the BM 34 may be configured to receive feedback from the patient.

Additionally, a separate device such as a wearable activity monitor (WAM) 50 may be used. The WAM 50 may be, for example, a smart watch or exercise monitoring device, and may provide signals to the IPG 10 or other remote devices such as the RC 32 and/or BM 34, related to the patient's activity state. The WAM 50 may include an accelerometer such as a 3-axis accelerometer for detecting activity. In some embodiments, the WAM 50 may include a heart rate monitor, gyroscope, global positioning system (GPS), and any other suitable sensors to determine and measure activity. The WAM 50 may instead or in addition provide other patient status data including, for example, monitoring blood constituents (such as glucose), breathing, body position (standing or lying prone), movement while in bed (sitting, rolling over), or monitoring for patient tremor, analyzing gait, etc. Rather than a wearable device, the WAM may instead take the form of an implantable device, if desired, such as a monitor (for example a cardiac monitor) or another therapy system (a drug pump, insulin pump, Vagus nerve stimulator, pacemaker, etc.).

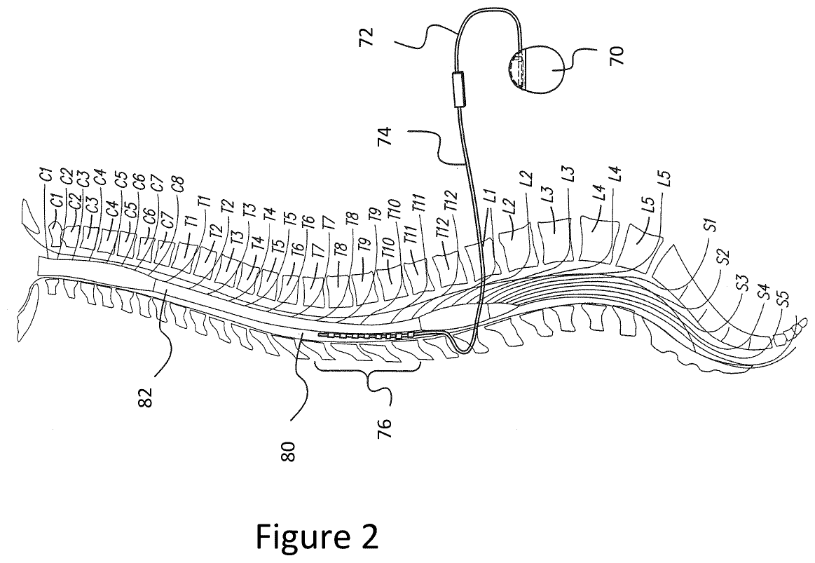

FIG. 2 shows an implantable spinal cord stimulation (SCS) system. Here, an IPG is shown at 70 and may be placed, for example, in the buttocks or torso of the patient, with a lead 74 extending into the spinal column 80 with a plurality of distal electrodes. Modulatory signals are generated to modulate pain signaling in the spinal cord 82, as is known in the art. The lead 74 may include a lead extension 72, as shown. The SCS system may include (not shown) any of the external or additional devices of FIG. 1, such as the CP 30, RC 32, BM 34, Charger 36, ETS 38, and/or WAM 50, as desired. While FIGS. 1 and 2 show illustrative implantable systems, other systems may also use the present inventive concepts, including cardiac systems (pacemakers, defibrillators, etc.), drug pumps, insulin pumps, neuromodulation systems (Vagus, sacral, etc.), and any system having two or more related devices to which communication may be established, particularly each of a home monitor and programmer or patient remote.

A system may be provided that performs a method of regulating data downloads from an IPG. The system and method may reduce potential interference between downloading data and calls for therapy. The system may include an IPG configured to download data, receive operation signals and provide electrical or other therapy to a patient, and at least a first external device configured to initiate a first data download from the IPG to the first external device. The first external device may be configured to direct the IPG to download the data, with a limit, such as for a first time period that is preset. The IPG and/or the first external device may be configured to stop the data download and terminate the communications session after the first preset time period has expired. After expiration of the first preset time period, the IPG and/or the first external device may be configured to wait a first time interval during which no data is downloaded and the IPG is available to receive signals from any external control devices. This pause in the downloading of data from the IPG to an external device allows for any other external device to request and initiate communication with the IPG, such as to initiate a therapeutic electrical signal to the patient. After the predetermined first waiting time interval has expired, the IPG and/or the first external device may initiate an additional data download from the IPG to the first external device for a second time period. At this point, one cycle of downloading data, stopping the download to wait, and re-initiating data download has occurred. The IPG and/or the first external device may be configured to repeat this cycle of initiating data download, stopping, waiting, and initiating additional data download steps until all data has been downloaded from the IPG to the first external device.

In some embodiments, the first external device may be configured to communicate with the IPG via a Bluetooth connection, and the first external device may be the RC (32) or BM (34) The IPG and/or another device in the system may be configured to measure activity level of the patient. For example, the IPG and/or the first external device may include an accelerometer. Alternatively, or in addition to the IPG and first external device, a wearable activity monitor (WAM 50 in FIG. 1, for example) may detect and measure activity of the patient. Example wearable activity monitors include smart watches, exercise monitors, heart rate monitors, GPS trackers, etc.

In some embodiments, the first time period during which the first data download occurs may be limited to a relatively short time period of between 30 seconds and 2 minutes. The data download may occur over a wireless connection, such as Bluetooth, between the IPG and the first external device. The second time period during which additional data downloads may occur may also be between 30 seconds and 2 minutes. The first and second time periods may be predetermined and may be the same or different, within the 30 second to 2 minute range; longer or shorter limits may be used if desired. In order to provide time for the IPG to be receptive to calls from other external devices, such as the RC, that may be attempting to initiate communication to send a signal to the IPG such as for therapeutic stimulation, the data download time periods may be staggered so they do not occur adjacently. A minimum time interval may separate the data download time periods. In some embodiments, the time interval between the first and second data download time periods is also 30 seconds to 2 minutes. In order to avoid confusion, the phrase time period refers to the time during which data is being downloaded from the IPG to an external device, and the phrase time interval refers to the wait time during which no data is downloaded and the IPG is available to receive calls or signals from any external device, for example for providing stimulation to the patient.

For the IPG and/or some external devices, such as cell phones, repeated Bluetooth connection may reduce battery life. When such a device is the first external device receiving the data downloads, the time periods during which data is downloaded and/or the time interval during which no data is downloaded may be extended, such as to two to five minutes. In other embodiments, a verification may be made that a secondary external device, capable of calling for or changing stimulation, is not trying to connect to the IPG. For example, such verification or monitoring may occur by checking for any connection or handshake requests on communication channels used for such requests in the relevant wireless protocol. In an example, if using Bluetooth Low Energy, the advertising channels in the spectrum may be monitored for a period of time (as would be the case in any event) to determine whether any connection requests are being made.

Either the IPG or the first external device may enforce the short time period for the type of connection needed for the data download. For example, the IPG and/or the first external device may be configured to set one or more timers to the first and second time periods and the first time interval. In some embodiments, at the start of a data download session, an indication may be provided to one or both of the IPG and first external device that the data download session will terminate in the first period of time. The session type may be defined by a message issued from one device to the other during the communication session, if multiple session types are allowed. For example, communication between an IPG and a remote monitor or BM may include each of data download (IPG communicating data to the BM) and/or a firmware update (BM communicating new firmware data to the IPG). Either the IPG or the first external device may terminate the data download session after the first time period has expired.

When the first external device is a cell phone, a mobile application may be used that is configured to prevent an additional data download session until the wait time interval timer expires. The IPG may prevent a data download session from the same external device for a period of time. For example, the IPG may receive communication advertisements, and may review an external device's tag or ID to determine if the same external device is requesting data download repeatedly. The data download session start request may indicate the session type, and the IPG may only allow certain types of sessions after an IPG download or read only session ends for a period of time.

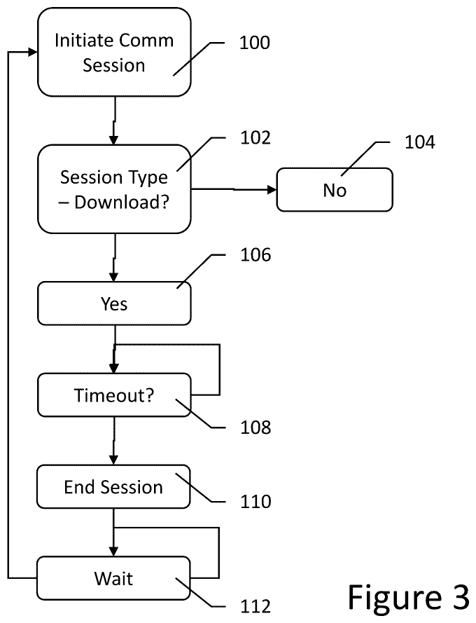

FIG. 3 shows an example. A communication session is initiated, such as using standard advertising and handshake protocols for the communication protocol, as indicated at 100. Optionally the session type may be determined at 102; if not a download of IPG data to a remote monitor, the method ends at 104 and simply uses standard communication steps to continue the session until the link is broken or the session termination. If a data download is occurring, as indicated at 106, the session is subject to a timeout at 108. When the timeout occurs, the session ends at 110. A wait period can then be enforced as shown at 112, before the next initiation occurs at 100. If the download session ends before the timeout 108, the method may simply skip to ending the session, and the wait period 112 may be optional if desired in such a scenario.

Another example of when data downloads from the IPG may interfere with aspects of the stimulation system is when automatic data downloads occur during periods when the patient is active. Attempting to download data when the patient is active may interfere with the stimulation system. For example, when the patient is active, the patient or the system itself may be expected to modulate stimulus from an internal command, such as from the IPG, or from an external command, such as from the RC. A method is provided for regulating data downloads from the IPG to an external device, in which automatic or requested data downloads are prevented when patient activity is occurring. The first step in this method is to detect and/or measure activity level of the patient. This may be achieved by using an accelerometer to measure movement. The accelerometer may be in the IPG or the external device such as a cell phone. In other embodiments, a wearable device that communicates with the IPG may be used. The wearable device may have an accelerometer or other motion or activity sensing technology, such as GPS or a heart rate monitor. Activity level may be measured continuously or periodically.

In some embodiments, detected activity must be above a predetermined threshold level before data download will be prevented. The threshold level of activity may be set by the patient, physician, or caregiver through an associated app, or it may be set and/or adjusted by the system based on previous download sessions. A relatively low level of measured activity may be due to the patient's physical symptoms and may not interfere with the data download. The system may be configured to monitor and record times of low level activity as measured by an accelerometer, and request confirmation by the patient that he or she was asleep. In this way, the system may set a threshold level of activity measured by an accelerometer, and this predetermined threshold level of activity may be used to determine how much “activity” is needed to trigger the method and prevent data downloads.

The IPG may detect activity directly through its accelerometer, if present. Alternatively, the RC may be in communication, either directly or indirectly, with another external device having an accelerometer or sensors for detecting activity, such as GPS or heart rate monitor. When the RC detects activity via accelerometer or other signals from an external device, the RC will communicate to the monitoring device requesting the data download to stop the request. Additionally, manual device interaction in a device capable of requesting an auto download from the IPG may be used as an indication of activity. Additionally, any device in direct communication or indirect communication, such as through a server, with the automated download devices may provide an indication of activity. Such a device may be the RC, cell phone, or bedside monitor. If the RC is a multi-function device, such as a cell phone, “activity” monitoring may also include monitoring for use of the cell phone, such as use of social media or the internet, or use of a music or podcast app, or use of a white noise generator that the patient relies on during sleep. Different responses may be designed for different uses; for example, if the patient is scrolling online or in a social media app, this may be treated as activity indicating no session should start; if the patient is instead listening to a podcast or has a white noise generator active, this may indicate the patient intends to sleep, be at rest, or remain stationary, and the session may initiate after a set interval of time from the start of the sleep or rest associated activity.

Various system interactions may occur after patient activity is determined and/or measured. In a first system interaction, the system will not initiate data download when any accelerometers are in communication, either directly or indirectly, with the IPG record activity. In a second system interaction, the system may check either directly with any activity-measuring device, including the RC device, or through a request made indirectly through a server or other means, if the activity-measuring device has recorded activity, either through an accelerometer or other means, within a preset prior interval. In some embodiments, the prior preset interval may be up to 30 minutes, or longer or shorter. In a third system interaction, upon first connection for an auto-download, the system will confirm if the IPG has recorded activity within a preset prior interval, which may be up to 30 minutes, or longer or shorter. When patient activity is found via any of the three system interactions above, auto download will be delayed for a predetermined period of time, such as up to 30 minutes, or longer or shorter.

In the event that the patient's motor symptoms make the IPG accelerometer unreliable for accurately detecting true activity versus disease related movement occurring during sleep, this detection method can be disabled. This may be disabled by the patient, physician, or caregiver, or automatically by the system if sufficient confidence is gained that the motion on the accelerometer is not reflective of “true” physical activity. If the external device with data download capability is unable to confirm a lack of activity from any activity-measuring device, the system may default to delaying or preventing data download until the lack of patient activity can be confirmed. Additionally, the IPG may terminate an automatic or requested data download in response to any error message anywhere in the system, or in responses to a detected event that may interfere with the data download. For example, the detected event may include physical symptoms detected by the IPG or an external device in communication with the system, or technical issues detected in any of the system components, including low battery charge or communications failure.

When patient activity above a predetermined threshold has been detected by any of the above methods, the system prevents data downloading from the IPG to any external devices. The method may involve preventing data downloading only when the activity level has been above the predetermined threshold for a first predetermined time period, for example at least 10-40 minutes. Once the data download has been prevented or stopped, the system is configured to wait a second period of time during which no data is downloaded and the IPG is available to receive signals from any external control devices. In some embodiments, the second period of time may be at least 10-40 minutes. For example, the first and second time periods may be at least 30 minutes. This second period of time allows for any patient activity to stop. After the second period of time expires, the activity level of the patient is measured again, and if activity is measure above the threshold, the data download continues to be prevented. If no activity is detected or the level of activity is below the threshold, then the system may initiate data downloading from the IPG to the external device that either automatically receives the data or requested it.

Another example of when data downloads from the IPG may interfere with aspects of the stimulation system is when automatic data downloads occur during periods when the patient is communicating with the IPG or using external devices that communicate with the IPG. If the patient is attempting to change their stimulation program, data downloads may interfere with the changes. A method is provided for regulating data downloads from the IPG to an external device, in which automatic data downloads occur when the patient is asleep and requested data downloads may be scheduled when the patient is expected to be asleep. The first step may be

determining if the patient is asleep, and automatically downloading data from the IPG to the first external device when the patient is asleep. Determining if the patient is asleep may be achieved by comparing a current time of day and/or day of the week with a sleep schedule set by the patient. The IPG and/or any external device within the connected system receiving the data download may have an internal clock, and the patient may upload a sleep schedule to the component of the system. In some embodiments, the component may be the IPG. Additionally, the component of the system receiving the sleep schedule may include calendar data including weekends and holidays, which may indicate later bedtimes, particularly when tracked over time. In other embodiments, the patient's sleep schedule may be determined and/or scheduled by the IPG or another external device, such as a cell phone or wearable device such as a fitness tracker. Determining the patient is asleep may be achieved by a lack of activity measured by an accelerometer. Another way the patient's wakefulness state may be determined is by detecting and/or measuring the patient's activity on or interaction with all known devices used by the patient that are capable of communicating with the IPG or another device in the system. The patient may provide a list of such devices, including a cell phone, wearable activity tracker, computer, tablet, etc. For example, a state of wakefulness is determined when the patient is actively using their cell phone, a computer or tablet that is in communication with the IPG, or the remote control.

Alternatively, or in addition to the above ways of determining the patient's wakefulness state, physiological parameters associated with sleep may be measured. For example, increased brain O2 use and/or increased and variable pulse and blood pressure may be measured as indicative of a sleep state. Further, if a patient has a scheduled pattern that has remained reasonably consistent across analysis to indicate sleep, the system may skip data downloads on a given night if pattern activity significantly deviates from what was established. Elements of the pattern may include bedtime and decrease in level of activity. In some embodiments, communication with or use of applications (apps) on a cell phone, computer, or tablet that indicate the patient is approaching a sleep state may be used. For example, if the patient has a history of using a meditation app or playing a specific playlist of music just prior to sleep, this information may be used as an indicator the patient is going to bed.

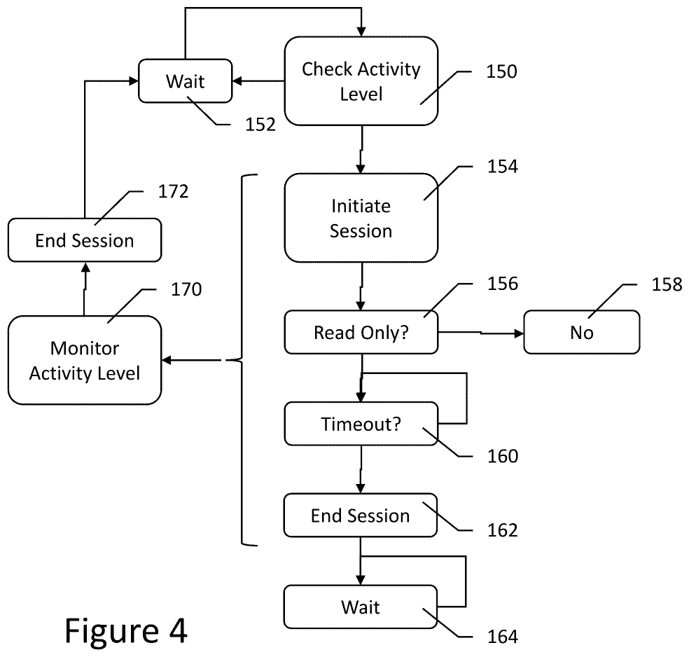

FIG. 4 shows an example which may be adapted to any of the preceding examples. Patient activity level is checked at 150; if the patient is active, a wait period is enforced at 152. Once the patient is inactive, a session can be initiated at 154. If a read-only session, such as downloading IPG data is not occurring, communication may proceed without the timeout and wait cycles, as described in FIG. 3. Optionally, the check for read-only at 156 may instead precede the patient activity check, allowing sessions that are not of certain types to proceed regardless of activity. If a timeout occurs at 160, the session ends at 162 and a wait state is enforced at 164. During the communication session, patient activity may be monitored as indicated at 170. If patient activity is detected, the session may be terminated as indicated at 172, and the wait enforced at 152, if desired.

Another example of when data downloads from the IPG may interfere with aspects of the stimulation system is when automatic data downloads occur during periods when the communication signal is weak. A method is provided for regulating data downloads from the IPG to an external device, in which automatic or requested data downloads are prevented when the communication signal between the IPG and the external device is weak. The communication signal may be Bluetooth, radiofrequency, or any other communication means. When the communication signal, particularly a Bluetooth signal, is weak, the data download may be interrupted and need to start over. Determination of signal strength may be performed using any suitable method, such as using the received signal strength indicator (RSSI) typically available with most devices. Signal strength may be determined by comparison to in-band noise if desired, such as by determining whether the frequency band to be used is itself noisy, as may happen if multiple users are present in the band of interest; if noise exceeds a threshold, or if the RSSI fails to exceed detected noise by a threshold amount (such as by about 3 to about 20 dB), the signal strength may be deemed weak.

The method may include preventing or stopping the initiation of the first data download for at least a period of time when the signal between the IPG and the external device has a strength below a predetermined level. After the period of time, if the signal is still below the predetermined level, then the system may stop any attempts to download data for 12 hours. If the data download has to restart a number of times within a set time period, the system may stop attempting to download data for an extended period of time. For example, after 2-5 instances of download failure within 5-10 minutes, the IPG or external device may cancel the data download for 5-12 hours. Further, if two consecutive attempts to connect are made 1 or more hours apart and both fail, the system may stop attempting to connect for a longer period of time, such as 24-36 hours. Such an approach may be used by analogy in FIG. 4 by replacing block 150 with “Check Signal Strength” and block 170 with “Monitor Signal Strength.”

In addition to stopping the data download when the signal strength is weak, the data download may be prevented or stopped for at least an additional period of time when a battery level of the IPG or the first external device is below a predetermined level. For example, when the IPG or external device battery is at less than 20%, data downloads may be stopped or prevented. When the data download is prevented or stopped after a predetermined number of attempts within a first predetermined period of time, the system may notify the patient and/or manufacturer of the system or its components. For example, if data download fails or is prevented more than 5 times within 5 hours, the patient may be notified. When a predetermined number of data downloads have been attempted during the additional period of time, then the system may limit the number and/or duration of future data downloads for a period of time. Again, this method may be incorporated into FIG. 4 by replacing block 150 with “Check Battery Level,” and block 170 with “Monitor Battery Level.”

In any of these examples, if a session cannot be initiated for one or another reason, the user/patient may be notified using known annunciation means (vibration or issuing tones for example) in the IPG, or by providing an alert via the patient RC or any other connected system component when it is next communicatively coupled to the IPG.

The above description is intended to be illustrative, and not restrictive. For example, the above-described examples (or one or more aspects thereof) may be used in combination with each other. Other embodiments can be used, such as by one of ordinary skill in the art upon reviewing the above description.

The Abstract is provided to comply with 37 C.F.R. § 1.72 (b), to allow the reader to quickly ascertain the nature of the technical disclosure. It is submitted with the understanding that it will not be used to interpret or limit the scope or meaning of the claims.

Also, in the above Detailed Description, various features may be grouped together to streamline the disclosure. This should not be interpreted as intending that an unclaimed disclosed feature is essential to any claim. Rather, innovative subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the protection should be determined with reference to the appended claims, along with the full scope of equivalents to which such claims are entitled.

What is claimed is:

1. A system for regulating data downloads from an implantable pulse generator (IPG), comprising:
an IPG configured to download data, receive operation signals and provide electrical stimulation to a patient; and
at least a first external device configured to initiate a first data download from the IPG to the first external device, the first external device configured to direct the IPG to download the data for a first time period, wherein the first time period is preset;
wherein the IPG and/or the first external device is configured to stop the data download after the first time period has expired;
wherein the IPG and/or the first external device is configured to wait a first time interval during which no data is downloaded and the IPG is available to receive signals from any external control devices;
wherein the IPG and/or the first external device is configured to initiate an additional data download from the IPG to the first external device for a second time period after the first time interval has expired;
wherein the IPG and/or the first external device is configured to repeat the initiating, stopping, waiting, and initiating additional data download steps until all data has been downloaded from the IPG to the first external device.

2. The system of claim 1, wherein the first external device is a remote control, bedside monitor, or other device with a monitoring application.

3. The system of claim 1, wherein the first external device is configured to communicate with the IPG via a Bluetooth connection.

4. The system of claim 1, wherein the IPG and/or the first external device is configured to measure activity level of the patient.

5. The system of claim 4, wherein the IPG and/or the first external device includes an accelerometer.

6. A method for regulating data downloads from an implantable pulse generator (IPG), comprising:
initiating a first data download from the IPG to a first external device and downloading data for a first time period, wherein the first time period is preset;
stopping the data download after the first time period has expired;
waiting a first time interval during which no data is downloaded and the IPG is available to receive signals from any external control devices;
after the first time interval has expired, then initiating an additional data download from the IPG to the first external device for a second time period;
repeating the initiating, stopping, waiting, and initiating additional data download steps until all data has been downloaded from the IPG to the first external device.

7. The method of claim 6, wherein the first external device is a remote control for the IPG, a bedside monitor, or other device with a monitoring application.

8. The method of claim 6, wherein the first external device is a cell phone.

9. The method of claim 6, wherein initiating the first and additional data downloads from the IPG to the first external device occurs over a Bluetooth connection.

10. The method of claim 6, wherein the first and second time periods are each between 30 seconds and two minutes.

11. The method of claim 6, wherein the first time interval is between 30 seconds and two minutes.

12. A method for regulating data downloads from an implantable pulse generator (IPG), comprising:
measuring an activity level of a patient;
preventing data download from the IPG to at least a first external device when the activity level has been above a threshold for a first period of time;
waiting a second period of time during which no data is downloaded and the IPG is available to receive signals from any external control devices;
measuring activity level of the patient; and
initiating data download from the IPG to the first external device when the activity level drops below the threshold.

13. The method of claim 12, wherein measuring activity level is achieved by an accelerometer or the IPG.

14. The method of claim 12, wherein measuring activity level is performed by a wearable device that is in communication, directly or indirectly, with the IPG.

15. The method of claim 12, further comprising determining if the patient is asleep, and automatically downloading data from the IPG to the first external device when the patient is asleep.

16. The method of claim 15, wherein determining if the patient is asleep is achieved by one or more of comparing a current time of day with a sleep schedule set by the patient, a lack of activity measured by an accelerometer, and measuring the patient's activity on or interaction with all known devices used by the patient.

17. The method of claim 12, wherein initiating the data download is prevented or stopped for at least a third period of time when a signal between the IPG and the first external device has a strength below a predetermined level.

18. The method of claim 17, wherein after the third period of time, if the signal is still below the predetermined level, then stop any attempts to download data for 12 hours.

19. The method of claim 15, wherein initiating the data download is prevented or stopped for at least a third period of time when a battery level of the IPG or the first external device is below a predetermined level.

20. The method of claim 17, wherein if the data download is prevented after a predetermined number of attempts within a first predetermined period of time, the method further comprises notifying the patient, physician, or caregiver.
